(19) United States
(12) Patent Application Publication
(10) Pub. No.: US 2022/0412932 A1
(43) Pub. Date: Dec. 29, 2022

(54) CHEMICAL INDICATOR STRIP FOR DISINFECTION

(71) Applicant: Sterifre Medical, Inc., Kirkland, WA (US)

(72) Inventors: Czeslaw Golkowski, Ithaca, NY (US); Parag Saraf, Kirkland, WA (US); Florgene Minor, Redmond, WA (US)

(21) Appl. No.: 17/848,833

(22) Filed: Jun. 24, 2022

Related U.S. Application Data
(60) Provisional application No. 63/215,218, filed on Jun. 25, 2021.

(51) Int. Cl. G01N 31/22; A61L 2/28; G01N 21/78; C09D 11/50; C09D 11/108; C09D 11/037; C09D 11/023

(52) U.S. Cl. CPC G01N 31/226; A61L 2/28; G01N 21/78; C09D 11/50; C09D 11/108; C09D 11/037; C09D 11/023

(57) ABSTRACT

A chemical indicator strip for detecting disinfectant that includes a film substrate, and an indicator portion with a first and a second ink. The first and the second ink can react in the presence of a threshold level of disinfectant for a threshold level of exposure time. The chemical indicator strip can be used in a method of detecting a threshold level of disinfectant. The method can include exposing a chemical indicator strip to a concentration of disinfectant for a preset amount of time. The method can include providing a visual indicator to a user that changes from a first color to a second color. The method can indicate that the chemical indicator strip has been exposed to a threshold concentration of disinfectant for a threshold amount of time when a complete color change occurs on the chemical indicator strip from the first color to the second color.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND

### Field

The disclosure relates to a chemical indicator for indicating exposure to a threshold level of disinfectant/sterilant. The chemical indicator can indicate exposure to more than one disinfectant/sterilant.

### Description of the Related Art

As disclosed herein, a variety of items or surfaces may require processing in order to reduce the bioburden and decrease risk of infections. For example, critical items (such as surgical instruments, which contact sterile tissue), semi-critical items (such as endoscopes, which contact mucous membranes), and noncritical items (such as stethoscopes, which contact only intact skin) require various types of treatment, for example sterilization, high-level disinfection, and low-level disinfection, respectively. Particularly in the medical field where medical instruments are used for the diagnosis and treatment of medical ailments. As transmission of microorganisms to a patient from a medical device can result in serious illness or death, the medical instrument should be free of microorganisms which thereby minimizes the spread of disease or infection to the patient. Medical devices can be sterilized using a sterilant and detecting the presence of an appropriate amount of sterilant can be desirable.

### SUMMARY

A method of sterilizing medical instruments can include providing sterilant or disinfectant in the form of liquid, gas, or gas-plasma to a sealed chamber housing the device to be disinfected or sterilized (e.g., medical instruments). Providing the sterilant in gas or plasma form (hereinafter “gas”) is especially desirable because the gas renders the surface of the device sterile, thereby rendering viruses and bacteria harmless. Likewise, the gas spreads to enclosed or isolated areas of the device which would otherwise not be sterilized. After a period of time, the sterilant in gas form renders the device sterile and the device can be removed from the chamber.

In order to verify that an adequate amount of disinfectant or sterilant was provided to the chamber, an indicator can be placed in the chamber with the device to be disinfected/sterilized. The indicator can include a chemical indicator or a biological indicator. A chemical indicator comprises a surface having a chemical thereon which changes color upon exposure to a sterilization process. Chemical indicators are often integrated with other sterilization verification devices to provide additional evidence of exposure to sterilant. Biological indicators are packages which contain a high number of bacterial spores. The biological indicator, after being exposed to sterilant, is culture tested to determine if the bacteria are viable. If the sterilization was successful, the bacteria contained within the biological indicator will not grow.

In general, chemical indicators should satisfy several basic performance characteristics. The chemical indicator should be readable, reliable, selective, stable, and safe. These performance characteristic are explained in more detail in Volume 1 of the Sterilization Standards Committee of the Association for the Advancement of Medical Instrumentation (AAMI) and by the General Requirements for Chemical Indicators of the proposed American National Standard Institute (ANSI) drafted by Chemical Indicators Working Group. These two documents are incorporated herein by reference.

Chemical indicators of the prior art generally comprise a pH-sensitive material placed on a medium. Simply, the pH-sensitive chemical indicator changes color when exposed to an acidic oxidation-type sterilant, such as H2O2 or H3CCOOOH, for a sufficient amount of time. The color change occurs from a chemically induced pH change, i.e., from basic to acid. For example, exposure to an acidic oxidation-type sterilant can change the color of a pH-sensitive chemical indicator from blue to yellow or colorless or from red to yellow. The pH-sensitive chemical indicators of the prior art can also be placed within containers known as test packs. A test pack is a structure which provides a challenge to the sterilization process and thereby provides a more realistic representation of actual conditions of certain areas on medical devices.

The pH-sensitive chemical indicators of the prior art suffer from several disadvantages and may not fully satisfy basic performance characteristics related to stability and selectivity. Since pH-sensitive chemical indicators are chemically reversible, their processed and unprocessed colors can change upon exposure to certain chemicals, especially those with acidic or basic characteristics. Poor pre-processing color stability of pH-sensitive chemical indicators of the prior art is undesirable because it requires chemical indicators to be discarded after a relatively short shelf life thereby wasting supplies. Furthermore, a chemical indicator of the prior art which is on the verge of changing color due to chemical instability does not provide an accurate chemical exposure indication when utilized in a sterilization chamber. Additionally, a short pre-processing shelf life for the chemical indicator is even more undesirable when the chemical indicator is integrated with a biological indicator because an even more expensive device (test pack, including chemical indicator and biological indicator) must be discarded when the pH-sensitive chemical indicator changes color.

Poor post-processing color stability of pH-sensitive chemical indicator of the prior art is also undesirable because the chemical indicator will return to its original color if exposed to a base and, thus, the processed chemical indicator cannot be used as a permanent record of the sterilization process. Therefore, if exposed to a base, the chemical indicator will revert to the original color and provide an unprocessed appearance. This characteristic of pH-sensitive chemical indicators of the prior art is particularly undesirable because using a chemical indicator more than once may provide a faulty processing indication. In a yet different scenario, the indicator could mislead a technician by providing an unprocessed reading when, in fact, the load was processed. This would lead to repeated sterilization cycles thereby increasing cost.

Finally, the pH-sensitive indicators of the prior art are not very selective and may change color upon exposure to any of a number of reagents, not just oxidation-type sterilants. Thus, an indicator may change color from exposure to an acidic reagent and not from adequate exposure to a sterilant. Such an inappropriate color change is misleading and could lead to misinterpretation of result.

Therefore, as disclosed herein, in order to verify that an adequate amount of disinfectant or sterilant was provided, an indicator can be used to indicate whether sufficient disinfected/sterilized was used in a method for disinfection or sterilization of a device. The method for disinfecting/sanitizing reduces the bioburden and decreases the risk of infections. The present disclosure provides for indicators for use in various systems and methods for disinfecting/sanitizing various items (e.g., medical devices or electronics) and surfaces (e.g., workspaces, patient rooms, organic material, including but not limited to patient wounds).

Various indicators are provided for herein in order to accomplish detecting whether threshold levels of disinfectant/sterilant was provided to one or more items, surfaces etc. Additionally, in several embodiments the indicators are configured to be used in systems and methods for low or high level disinfection. In still additional embodiments, the systems and methods are configured to allow sterilization.

In several embodiments, there is provided a chemical indicator strip comprising, consisting of, or consisting essentially of a IEVG-02 ink and a IORC-01 ink. In some embodiments, the IEVG-02 ink includes: styrene resin with a wt % of one of 60% wt, 65% wt, 70% wt, 75% wt, 80% wt, between 60%-70% wt, between 70%-80% wt, or between 60%-80% wt; water with a wt % of one of 5% wt, 6% wt, 7% wt, 8% wt, 9% wt, 10% wt, 11% wt, 12% wt, between 5%-9% wt, between 9%-15% wt; petroleum defoamer with a wt % of one of 0.50% wt, 1% wt, 1.50% wt, between 0.50%-1.0% wt, between 1.0%-1.5% wt, or between 1.5%-2.0% wt; ammonia with a wt % of one of 1% wt, 5% wt, 10% wt, or between 5%-10% wt; phenol red with a wt % of one of 0.50% wt, 1% wt, 1.50% wt, 2.0% wt, between 0.50%-1.0% wt, between 1.0%-1.5% wt, or between 1.5%-2.0% wt; common yellow pigment with a wt % of one of 1% wt, 1.50% wt, 2.0% wt, 2.50% wt, between 1%-1.50% wt, between 1.5%-2.0% wt, between 2.0%-2.50% wt; common blue pigment with a wt % of one of 0.50% wt, 1% wt, 1.50% wt, 2.0% wt, between 0.50%-1.0% wt, between 1.0%-1.5% wt, between 1.5%-2.0% wt; and quaternary ammonium bromide with a wt % of one of 0.50% wt, 1% wt, 1.50% wt, 2.0% wt, between 0.50%-1.0% wt, between 1.0%-1.5% wt, between 1.5%-2.0% wt. In some embodiments, the IORC-01 ink includes styrene acrylic emulsion resin with a wt % of one of 50% wt, 60% wt, 70% wt, 80% wt, 90% wt, between 50%-60% wt, between 60%-70% wt, between 70%-80% wt, between 80%-90% wt; water with a wt % of one of 8% wt, 10% wt, 12% wt, 14% wt, 16% wt, 18% wt, 20% wt, between 5%-10% wt, between 10%-15% wt, between 15%-20% wt; rosolin acid with a 1% wt, 2% wt, 3% wt, 4% wt, 5% wt, 6% wt, 7% wt, 8% wt, 9% wt, 10% wt, between 1%-3% wt, between 3%-5% wt, between 5%-7% wt, between 7%-10% wt, between 0%-5% wt, between 5%-10% wt; and petroleum defoamer with a wt % of one of 0.50% wt, 1% wt, 1.50% wt, 2.0% wt, between 0.50%-1.0% wt, between 1.0%-1.5% wt, between 1.5%-2.0% wt.

For example, provided for herein are several embodiments of a chemical indicator strip for detecting disinfectant. In some embodiments, the chemical indicator strip comprises a film substrate, an indicator portion comprising a first ink and a second ink, and wherein the first ink and the second ink react in the presence of a threshold level of disinfectant for a threshold level of exposure time. In some embodiments, the first ink and the second ink are water based resin carriers. In some embodiments, the first ink and the second ink include an acrylic emulsion, water, reactive pigments, a non-reactive colorant, and a defoaming agent.

In some embodiments, the first ink is IEVG-02. In some embodiments, the IEVG-02 comprises a styrene resin, water, petroleum, ammonia, phenol red pigment, common yellow pigment, common blue pigment, and quaternary ammonium bromide. In some embodiments, the styrene resin comprises between a 60% wt-80% wt of the IEVG-02. In some embodiments, the water comprises between a 1% wt-15% wt of the IEVG-02. In some embodiments, the petroleum defoamer comprises between 0.5% wt-0 1.0% wt, between 1.0% wt-1.5% wt, or between 1.5% wt-0 2.0% wt of the IEVG-02. In some embodiments, the ammonia comprises between 0% wt-5% wt or between 5% wt-10% wt of the IEVG-02. In some embodiments, the phenol red pigment comprises between 0.5% wt-0 1.0% wt of the IEVG-02, wherein the common yellow pigment comprises between 1% wt-5% wt of the IEVG-02, and wherein the common blue pigment comprises between 1.0% wt-0 1.5% wt of the IEVG-02. In some embodiments, the quaternary ammonium bromide comprises between 6% wt-8% wt of the IEVG-02. In some embodiments, the IEVG-02 comprises styrene resin with a 70% wt, water with a 12% wt, petroleum with a 1.5% wt, ammonia with a 4% wt, phenol red pigment with a 1% wt, common yellow pigment with a 3% wt, common blue pigment with a 1% wt, and a quaternary ammonium bromide with a 7.5% wt.

In some embodiments, the second ink is IORC-01. In some embodiments, the IORC-01 comprises styrene acrylic emulsion resin, water, rosolin acid, and petroleum defoamer. In some embodiments, the styrene acrylic emulsion resin comprises between 80% wt-90% wt of the IORC-01, wherein the water comprises between 5% wt-10% wt of the IORC-01, wherein the rosolin acid comprises between 0% wt-5% wt of the IORC-01, and wherein the petroleum defoamer comprises between 0% wt-2% wt of the IORC-01. In some embodiments, IORC-01 comprises styrene acrylic emulsion resin with a 88.5% wt, water with a 8.5% wt, rosolin acid with a 1% wt, and petroleum defoamer with a 1% wt.

In some embodiments, the film substrate comprises a single layer of biaxially oriented polypropylene. In some embodiments, the film substrate comprises an adhesive to allow the chemical indicator strip to attach to a surface. In some embodiments, the chemical indicator strip has a length between 2-4 inches and a width of between 0.75-1 inches. In some embodiments, the indicator portion is configured to change from a first color to a second color. In some embodiments, the first color is red or orange. In some embodiments, the second color is green. In some embodiments, the indicator portion is positioned at a first end of the chemical indicator strip. In some embodiments, the indicator portion is a square. In some embodiments, the indicator portion completely changes from the first color to the second color when the threshold level of disinfectant is detected. In some embodiments, the indicator portion does not change, or only partially changes from the first color to the second color when the threshold level of disinfectant is not detected. In some embodiments, the disinfectant detected is hydrogen peroxide and/or ozone. In some embodiments, the threshold level of exposure time is between 5 minutes and 10 minutes.

For example, provided for herein are several embodiments of a method of manufacturing a chemical indicator strip. In some embodiments, the method includes printing a first ink onto a film substrate. In some embodiments, the method includes allowing the first ink to dry on the film substrate. In some embodiments, the method includes printing a second ink on top of the first ink. In some embodiments, the method includes allowing the second ink to dry.

In some embodiments, the first ink and the second ink in the method of manufacturing are water based resin carriers. In some embodiments, the first ink and the second ink include an acrylic emulsion, water, reactive pigments, a non-reactive colorant, and a defoaming agent. In some embodiments, the first ink is IEVG-02. In some embodiments, the IEVG-02 comprises a styrene resin, water, petroleum, ammonia, phenol red pigment, common yellow pigment, common blue pigment, and quaternary ammonium bromide. In some embodiments, the second ink is IORC-01. In some embodiments, the IORC-01 comprises styrene acrylic emulsion resin, water, rosolin acid, and petroleum defoamer. In some embodiments, the film substrate comprises a single layer of biaxially oriented polypropylene.

In some embodiments, the method of manufacturing further comprises providing the film substrate with an adhesive surface to allow the chemical indicator strip to attach to a surface.

For example, provided for herein are several embodiments of a method for detecting a threshold level of disinfectant. In some embodiments, the method comprises exposing a chemical indicator strip to a concentration of disinfectant for a preset amount of time. In some embodiments, the method comprises providing a visual indicator to a user, wherein the visual indicator comprises at least a portion of the chemical indicator strip that changes from a first color to a second color. In some embodiments, in the method for detecting a threshold level of disinfectant, a complete color change from the first color to the second color indicates to the user that the chemical indicator strip has been exposed to a threshold concentration of disinfectant for a threshold amount of time.

In some embodiments, in the method for detecting a threshold level of disinfection, the chemical indicator strip is configured to detect threshold levels of hydrogen peroxide and ozone. In some embodiments, the visual indicator changes from a red to a green color. In some embodiments, the method for detecting a threshold level of disinfection uses a chemical indicator strip that comprises a first ink and a second ink. In some embodiments, the first ink comprises IEVG-02 and the second ink comprises IORC-01. In some embodiments, the IEVG-02 comprises a styrene resin, water, petroleum, ammonia, phenol red pigment, common yellow pigment, common blue pigment, and quaternary ammonium bromide. In some embodiments, the IORC-01 comprises styrene acrylic emulsion resin, water, rosolin acid, and petroleum defoamer.

In some embodiments, on the chemical indicator strip used in the method for detecting a threshold level of disinfection, exposure to hydrogen peroxide causes a first reaction with the quaternary ammonium bromide in the IEVG-02 ink that produces HBR, wherein the HBR creates a redox reaction that impacts the phenol red pigment in the IEVG-02 ink to produce a colorless end color, and wherein this can allow the common yellow pigment and the common blue pigment in the IEVG-02 ink to show a green color.

In some embodiments, on the chemical indicator strip used in the method for detecting a threshold level of disinfection, exposure to ozone causes a second reaction that that oxidizes the rosolin acid in the IORC-01 ink, and wherein the initial color of the rosolin acid is a red or orange color and becomes colorless once oxidized.

In some embodiments, in the method for detecting a threshold level of disinfection, the first reaction and the second reaction causes the chemical indicator to change into the green color. In some embodiments, in the method for detecting a threshold level of disinfection, the threshold amount of time is between 5 minutes and 10 minutes.

For example, provided for herein are several embodiments of a method of detecting hydrogen peroxide and ozone as substantially as shown and/or described herein.

For example, provided for herein are several embodiments of a method of preparing a chemical indicator strip as substantially as shown and/or described herein.

For example, provided for herein are several embodiments of a chemical indicator strip for detecting hydrogen peroxide and ozone substantially as shown and/or described herein.

For example, provided for herein are several embodiments of a chemical indicator strip substantially as shown and/or described herein.

For example, provided for herein are several embodiments of a method of detecting exposure time to hydrogen peroxide and ozone as substantially as shown and/or described herein.

For example, provided for herein are several embodiments of a chemical indicator strip for detecting exposure time to hydrogen peroxide and ozone substantially as shown and/or described herein.

### BRIEF DESCRIPTION OF THE DRAWINGS

These and other features, aspects and advantages are described below with reference to the drawings, which are intended for illustrative purposes and should in no way be interpreted as limiting the scope of the embodiments. Furthermore, various features of different disclosed embodiments can be combined to form additional embodiments, which are part of this disclosure. In the drawings, like reference characters denote corresponding features consistently throughout similar embodiments. The following is a brief description of each of the drawings.

FIG. 1A illustrates an embodiment of a cycle or process indicator strip with a color-changing indicator. FIG. 1A illustrates the cycle or process indicator strip before it has been used to test whether threshold levels of disinfectant/sterilant have been achieved.

FIG. 1B illustrates the cycle or process indicator strip of FIG. 1A wherein the color-changing indicator indicates that threshold levels of disinfectant/sterilant have not been achieved.

FIG. 1C illustrates the cycle or process indicator strip of FIG. 1A wherein the color-changing indicator indicates that threshold levels of disinfectant/sterilant have been achieved.

FIG. 2A illustrates another embodiment of a cycle or process indicator strip with a color-changing indicator. FIG. 2A illustrates the cycle or process indicator strip before it has been used to test whether threshold levels of disinfectant/sterilant have been achieved or if threshold levels of disinfectant/sterilant have not been achieved.

FIG. 2B illustrates the cycle or process indicator strip of FIG. 2A wherein the color-changing indicator indicates that threshold levels of disinfectant/sterilant have been achieved.

### DETAILED DESCRIPTION

The present disclosure describes an indicator for use in methods and systems for disinfection/sterilization. The disclosed cycle or process indicator (“CI”) strips can be used as an indicator when placed in a chamber with an item for disinfection or sterilization. When the CI is placed in the chamber for disinfection/sterilization, the CI can notify the user whether the items for disinfection/sterilization has been exposed to necessary levels of disinfectant/sterilant. In some embodiments, the CI can indicate to the user whether the item(s) for disinfection/sterilization has been exposed to necessary levels of H₂O₂ and/or ozone (e.g., activated oxygen).

In some embodiments, the CI can indicate whether a threshold level of disinfectant/sterilant by changing color. For example, the CI can include a colored box that changes from a first color to a second color when threshold levels of disinfectant/sterilant are detected. In some embodiments, the CI strip can include a box that changes from an red and/or orange to a green color when the threshold for H₂O₂ and/or ozone (e.g., activated oxygen) has been reached.

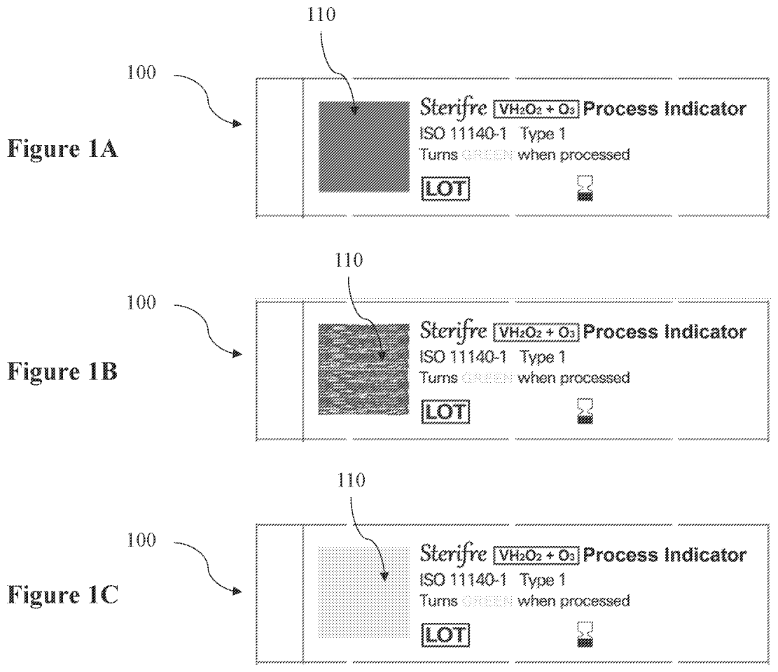

FIGS. 1A-1C illustrate an example of a CI strip including a color-changing indicator. FIG. 1A illustrates the CI strip 100 before use. The CI strip 100 can include an indicator 110. As shown in FIG. 1A, the indicator 110 of the CI strip 100 can be positioned on a first end of the CI strip 100. The indicator 110 illustrated in FIG. 1A is in the shape of a square, but the indicator 110 can be any shape or size that is visible to the user. The indicator 110 can be configured to change colors when exposed to threshold levels of disinfectant/sterilant. For example, the indicator 110 can change from a red/orange color to a green color when exposed to threshold levels of disinfectant/sterilant. The indicator 110 can change from any first color or design to any second color or design that would indicate to a user that the CI strip 100 has detected threshold levels of sterilant/disinfectant. In some embodiments, the CI strip 100 will indicate to the user what change to expect when threshold levels of the sterilant/disinfectant are detected. In the non-limiting example illustrated in FIGS. 1A-1C, the CI strip 100 indicates that when threshold levels of H₂O₂ and ozone (e.g., activated oxygen) have been met, the CI strip 100 “Turns GREEN when processed.”

FIG. 1A illustrates the CI strip 100 where the color indicator 110 indicates that threshold levels of disinfectant/sterilant have not been detected FIG. 1B illustrates the CI strip 100 where the color indicator 110 indicates that threshold levels of disinfectant/sterilant have not been detected. As shown, although the indicator 110 has minor color changes (e.g., some green on the initial red/orange color), the indicator 110 has not changed completely to the second green color. The lack of a complete color change of the indicator 110 on the CI strip 100 indicates to the user that the levels of sterilant/disinfectant have not met threshold values. This can indicate to the user, for example, that the item to be sterilized/disinfected may not have been properly sterilized/disinfected as the item has not been exposed to the requisite concentration of sterilant/disinfectant. In the example illustrated in FIG. 1B, the color change of the indicator 110 indicates to the user that the item to be sterilized/disinfected has not been exposed to the threshold levels of H₂O₂ and ozone (e.g., activated oxygen).

FIG. 1C illustrates the CI strip 100 where the color indicator 110 indicates that threshold levels of disinfectant/sterilant has been detected. As shown, the indicator 110 has change completely from the first red/orange color to the second green color. The complete color change of the indicator 110 on the CI strip 100 indicates to the user that the levels of sterilant/disinfectant has met the threshold value. This can indicate to the user, for example, that the item to be sterilized/disinfected has been properly sterilized/disinfected as the item has been exposed to the requisite concentration of sterilant/disinfectant. In the example illustrated in FIG. 1C, the color change of the indicator 110 indicates to the user that the item to be sterilized/disinfected has been exposed to the threshold levels of H₂O₂ and ozone (e.g., activated oxygen).

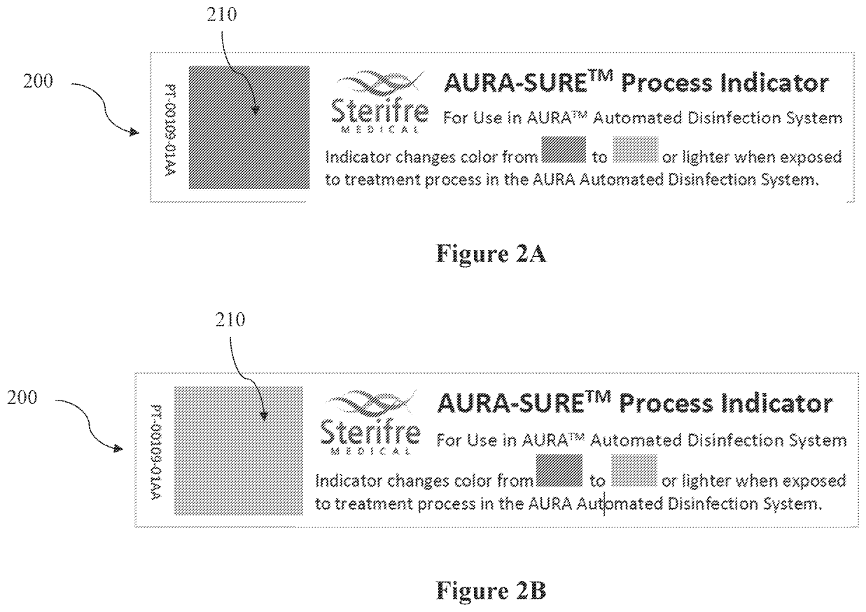

FIGS. 2A-2B illustrate another example of CI strip. Like the CI strip of FIGS. 1A-1C, the CI strip includes a color changing indicator. FIG. 2A illustrates the CI strip 200 before use. The CI strip 200 can include an indicator 210. As shown in FIG. 2A, the indicator 210 of the CI strip 200 can be positioned on a first end of the CI strip 200. The indicator 210 illustrated in FIG. 2A is in the shape of a square, but the indicator 210 can be any shape or size that is visible to the user. The indicator 210 can be configured to change colors when exposed to threshold levels of disinfectant/sterilant. For example, the indicator 210 can change from a red/orange color to a green color when exposed to threshold levels of disinfectant/sterilant. The indicator 210 can change from any first color or design to any second color or design that would indicate to a user that the CI strip 200 has detected threshold levels of sterilant/disinfectant. In some embodiments, the CI strip 200 will indicate to the user what change to expect when threshold levels of sterilant/disinfectant are detected. In the non-limiting example illustrated in FIGS. 2A-2B, the CI strip 200 indicates that when threshold levels of H₂O₂ and ozone (e.g., activated oxygen) have been met, the indicator 210 of the CI strip 200 “changes color from” a first color to a second color “or lighter when exposed to treatment process in the” disinfection system.

FIGS. 2B illustrates the CI strip 200 where the color indicator 210 indicates that threshold levels of disinfectant/sterilant has been detected. As shown, the indicator 210 can change completely from the first red/orange color to the second green color.

In some embodiments, the complete color change of the indicator 210 on the CIP strip 200 indicates to the user that the levels of sterilant/disinfectant has reached the threshold value. This can indicate to the user, for example, that the item to be sterilized/disinfected has been properly sterilized/disinfected as the item has been exposed the requisite concentration of sterilant/disinfectant. In the example illustrated in FIG. 2B, the color change of the indicator 210 indicates to the user that the item to be sterilized/disinfected has been exposed to the threshold levels of H₂O₂ and ozone (e.g., activated oxygen).

### Materials and Dimensions for CI Strip

In some embodiments, the CI strips can include a film substrate. In some embodiments, the film substrate is a single layer of biaxially oriented polypropylene (BOPP). In some examples, the film substrate can include an adhesive. The adhesive can allow the CI strips to be attached like a sticker to various surfaces (e.g., a log book).

In some embodiments, the CI strip can be rectangular, though any other geometric shape that allows exposure to the sterilant can be used. In those embodiments employing a rectangular shape, the CI strip can have a width of 1″ and a length of 341 . In some embodiments, the CI strip can have a width of 0.85″ and a length of 4″. In some examples, the CI strip can have a width of 0.75″ and a length of 2.75″. In some embodiments, the CI strip can have a width of 0.50″, 0.55″, 0.60″, 0.65″, 0.70″, 0.75″, 0.80″, 0.85″, 0.90″, 0.95″, 1.0″, 1.05″, 1.10″, 1.15″, 1.20″, 1.25″, between 0.50″-0.75″, between 0.75″-1.041 , between 1.0″-1.25″, and any value in between those ranges listed, including endpoints. In some examples, the CI strip can have a length of 2.50″, 2.55″, 2.60″, 2.65″, 2.70″, 2.75″, 2.80″, 2.85″, 2.90″, 2.95″, 3.0″, 3.05″, 3.10″, 3.15″, 3.20″, 3.25″, 3.30″, 3.35″, 3.40″, 3.45″, 3.55″, 3.60″, 3.65″, 3.70″, 3.75″, 3.80″, 3.85″, 3.90″, 3.95″, 4.0″ between 2.50″-2.75″, between 2.75″-3.0″, between 3.0″-3.25″, between 3.25″-3.50″, between 3.50″-3.75″, between 3.75″-4.0″, and any value in between those ranges listed, including endpoints. In some examples, the ratio between the width and the length of the CI strip is 1:2, 1:3, 3:11, 1:4, between 1:3-1:2, between 1:4-1:3, between 1:5-1:4, and any value in between those ranges listed, including endpoints.

### Ink for Use in CI Strip

The CI strip can be printed with a plurality of inks. For example, the CI strip can include a IEVG-02 ink and a IORC-01 ink. In some embodiments, the CI strip includes a IEVG-02 ink and a IORO-01 ink. The two inks can be made of a water based resin carrier. The carrier can include an acrylic emulsion, water, reactive pigments, a non-reactive colorant, and a defoaming agent. In some embodiments, the defoaming agent can prevent bubbles from forming when on press. In some examples, when used on card stock, we use ink that includes a BOPP substrate married to polylith card stock as both are polymers.

### IEVG-02 Ink

As discussed above, the CI strip can be printed with an IEVG-02 ink. In some embodiments, the IEVG-02 ink can include a combination of the following ingredients: styrene resin, water, petroleum defoamer, ammonia, phenol red, common yellow pigment, common blue pigment, and quaternary ammonium bromide.

In some embodiments, the IEVG-02 ink can include a styrene resin. The styrene resin can comprise a 60% wt, 62% wt, 64% wt, 66% wt, 68% wt, 70% wt, 72% wt, 74% wt, 76% wt, 78% wt, 80% wt, between 60%-65% wt, between 65%-70% wt, between 70%-75% wt, between 75%-80% wt, between 60%-70% wt, between 70%-80% wt, between 60%-80% wt, and any value in between those ranges listed, including endpoints.

In some embodiments, the IEVG-02 ink can include water. The water can comprise a 1% wt, 2% wt, 3% wt, 4% wt, 5% wt, 6% wt, 7% wt, 8% wt, 9% wt, 10% wt, 11% wt, 12% wt, 13% wt, 14% wt, 15% wt, between 1%-5% wt, between 5%-9% wt, between 9%-15% wt, between 1%-15% wt, and any value in between those ranges listed, including endpoints.

In some embodiments, the IEVG-02 ink can include a petroleum defoamer. The petroleum defoamer can comprise a 0.10% wt, a 0.20% wt, a 0.30% wt, a 0.40% wt, a 0.50% wt, a 0.60% wt, a 0.70% wt, a 0.80% wt, a 0.90% wt, a 1% wt, a 1.10% wt, a 1.20% wt, a 1.30% wt, a 1.40% wt, a 1.50% wt, a 1.60% wt, a 1.70% wt, a 1.80% wt, a 1.90% wt, a 2.0% wt, between 0%-0.50% wt, between 0.50%-1.0% wt, between 1.0%-1.5% wt, between 1.5%-2.0% wt, and any value in between those ranges listed, including endpoints.

In some embodiments, the IEVG-02 ink can include ammonia. The ammonia can comprise 1% wt, 2% wt, 3% wt, 4% wt, 5% wt, 6% wt, 7% wt, 8% wt, 9% wt, 10% wt, between 1%-3% wt, between 3%-5% wt, between 5%-7% wt, between 7%-10% wt, between 0%-5% wt, between 5%-10% wt, and any value in between those ranges listed, including endpoints.

In some embodiments, the IEVG-02 ink can include phenol red. The phenol red can comprise a 0.10% wt, a 0.20% wt, a 0.30% wt, a 0.40% wt, a 0.50% wt, a 0.60% wt, a 0.70% wt, a 0.80% wt, a 0.90% wt, a 1% wt, a 1.10% wt, a 1.20% wt, a 1.30% wt, a 1.40% wt, a 1.50% wt, a 1.60% wt, a 1.70% wt, a 1.80% wt, a 1.90% wt, a 2.0% wt, between 0%-0.50% wt, between 0.50%-1.0% wt, between 1.0%-1.5% wt, between 1.5%-2.0% wt, and any value in between those ranges listed, including endpoints.

In some embodiments, the IEVG-02 ink can include common yellow pigment. The common yellow pigment can comprise a 1% wt, 1.10% wt, 1.20% wt, 1.30% wt, 1.40% wt, 1.50% wt, 1.60% wt, 1.70% wt, 1.80% wt, 1.90% wt, 2.0% wt, 2.10% wt, 2.20% wt, 2.30% wt, 2.40% wt, 2.50% wt, 2.60% wt, 2.70% wt, 2.80% wt, 2.90% wt, 3.0% wt, 3.1% wt, 3.2% wt, 3.3% wt, 3.4% wt, 3.5% wt, 3.6% wt, 3.7% wt, 3.8% wt, 3.9% wt, 4.0% wt, 4.1% wt, 4.2% wt, 4.3% wt, 4.4% wt, 4.5% wt, 4.6% wt, 4.7% wt, 4.8% wt, 5.0% wt, between 1%-1.50% wt, between 1.5%-2.0% wt, between 2.0%-2.50% wt, between 2.50%-3.0% wt, between 1%-2% wt, between 2%-3% wt, between 3%-4% wt, between 4%-5% wt, between 1%-5%, and any value in between those ranges listed, including endpoints.

In some embodiments, the IEVG-02 ink can include common blue pigment. The common blue pigment can comprise a 0.10% wt, a 0.20% wt, a 0.30% wt, a 0.40% wt, a 0.50% wt, a 0.60% wt, a 0.70% wt, a 0.80% wt, a 0.90% wt, a 1% wt, a 1.10% wt, a 1.20% wt, a 1.30% wt, a 1.40% wt, a 1.50% wt, a 1.60% wt, a 1.70% wt, a 1.80% wt, a 1.90% wt, a 2.0% wt, between 0%-0.50% wt, between 0.50%-1.0% wt, between 1.0%-1.5% wt, between 1.5%-2.0% wt, and any value in between those ranges listed, including endpoints.

In some embodiments, the IEVG-02 ink can include quaternary ammonium bromide. The quaternary ammonium bromide can comprise a 0.10% wt, a 0.20% wt, a 0.30% wt, a 0.40% wt, a 0.50% wt, a 0.60% wt, a 0.70% wt, a 0.80% wt, a 0.90% wt, a 1% wt, a 1.10% wt, a 1.20% wt, a 1.30% wt, a 1.40% wt, a 1.50% wt, a 1.60% wt, a 1.70% wt, a 1.80% wt, a 1.90% wt, a 2.0% wt, a 3.0% wt, a 3.1% wt, 3.2% wt, a 3.3% wt, a 3.4% wt, a 3.5% wt, a 3.6% wt, a 3.7% wt, a 3.8% wt, a 3.9% wt, a 3.9% wt, a 4.0% wt, a 4.1% wt, a 4.2% wt, a 4.3% wt, a 4.4% wt, a 4.5% wt, a 4.6% wt, a 4.7% wt, a 4.8% wt, a 4.9% wt, a 5.0% wt, a 5.1% wt, a 5.2% wt, a 5.3% wt, a 5.4% wt, a 5.5% wt, a 5.6% wt, a 5.7% wt, a 5.8% wt, a 5.9% wt, a 6.0% wt, a 6.1% wt, a 6.2% wt, a 6.3% wt, a 6.4% wt, a 6.5% wt, a 6.6% wt, a 6.7% wt, a 6.8% wt, a 6.9% wt, a 7.0% wt, a 7.1% wt, a 7.2% wt, a 7.3% wt, a 7.4% wt, a 7.5% wt, a 7.6% wt, a 7.7% wt, a 7.8% wt, a 7.9% wt, a 8.0% wt, between 0%-0.50% wt, between 0.50%-1.0% wt, between 1.0%-1.5% wt, between 1.5%-2.0% wt, between 2.0%-2.5% wt, between 2.5%-3.0% wt, between 3.0%-3.5% wt, between 3.5%-4.0% wt, between 4.0%-4.5% wt, between 4.5%-5.0% wt, between 5.0%-5.5% wt, between 5.5%-6.0% wt, between 6.0%-6.5% wt, between 6.5%-7.0% wt, between 7.0%-7.5% wt, between 7.5%-8.0% wt, between 0%-2% wt, between 2%-4% wt, between 4%-6% wt, between 6%-8% wt, and any value in between those ranges listed, including endpoints.

In some embodiments, the IEVG-02 ink comprises a 70% wt of styrene resin, a 12% wt of water, a 1.5% wt of petroleum, a 4% wt of ammonia, a 1% wt of phenol red pigment, a 3% wt of common yellow pigment, a 1% wt of common blue pigment, a 7.5% wt of quaternary ammonium bromide.

In some examples, to prepare the IEVG-02 ink, the components of the IEVG-02 ink are added into a pail and mixed together for a minimum of one hour. Following mixing, the IEVG-02 ink can be filtered through a 250 micron filter into a clean container.

### IORC-01 Ink

The CI strip can be printed with an IORC-01 ink. In some embodiments, the IORC-01 ink can include a combination of the following ingredients: styrene acrylic emulsion resin, water, rosolin acid, and petroleum defoamer.

In some embodiments, the IORC-01 ink can include a styrene acrylic emulsion resin. The styrene acrylic emulsion resin can comprise a 50% wt, 55% wt, 60% wt, 65% wt, 70% wt, 75% wt, 80% wt, 85% wt, 90% wt, between 50%-60% wt, between 60%-70% wt, between 70%-80% wt, between 80%-90% wt, and any value in between those ranges listed, including endpoints.

In some embodiments, the IORC-01 ink can include water. Water can comprise 2% wt, 4% wt, 6% wt, 8% wt, 10% wt, 12% wt, 14% wt, 16% wt, 18% wt, 20% wt, 22% wt, 24% wt, 26% wt, 28% wt, 30% wt, between 2%-5% wt, between 5%-10% wt, between 10%-15% wt, between 15%-20% wt, between 20%-25% wt, between 25%-30% wt, and any value in between those ranges listed, including endpoints.

In some embodiments, the IORC-01 ink can include rosolin acid. The rosolin acid can comprise 0.1% wt, 0.2% wt, 0.3% wt, 0.4% wt, 0.5% wt, 0.6% wt, 0.7% wt, 0.8% wt, 0.9% wt, 1% wt, 2% wt, 3% wt, 4% wt, 5% wt, 6% wt, 7% wt, 8% wt, 9% wt, 10% wt, between 0%-1% wt, between 1%-3% wt, between 3%-5% wt, between 5%-7% wt, between 7%-10% wt, between 0%-5% wt, between 5%-10% wt, and any value in between those ranges listed, including endpoints.

In some embodiments, the IORC-01 ink can include a petroleum defoamer. The petroleum defoamer can comprise a 0.10% wt, a 0.20% wt, a 0.30% wt, a 0.40% wt, a 0.50% wt, a 0.60% wt, a 0.70% wt, a 0.80% wt, a 0.90% wt, a 1% wt, a 1.10% wt, a 1.20% wt, a 1.30% wt, a 1.40% wt, a 1.50% wt, a 1.60% wt, a 1.70% wt, a 1.80% wt, a 1.90% wt, a 2.0% wt, between 0%-0.50% wt, between 0.50%-1.0% wt, between 1.0%-1.5% wt, between 1.5%-2.0% wt, between 0%-2.0% wt, and any value in between those ranges listed, including endpoints.

In some examples, to prepare the IORC-01 ink, the components of the IORC-0 link are added into a pail and mixed together for a minimum of one hour. Following mixing, the IORC-0 link can be filtered through a 250 micron filter into a clean container.

In some embodiments, the IEVG-02 ink includes: styrene resin with a wt % of one of 60% wt, 65% wt, 70% wt, 75% wt, 80% wt, between 60%-70% wt, between 70%-80% wt, or between 60%-80% wt; water with a wt % of one of 5% wt, 6% wt, 7% wt, 8% wt, 9% wt, 10% wt, 11% wt, 12% wt, between 5%-9% wt, between 9%-15% wt; petroleum defoamer with a wt % of one of 0.50% wt, 1% wt, 1.50% wt, between 0.50%-1.0% wt, between 1.0%-1.5% wt, or between 1.5%-2.0% wt; ammonia with a wt % of one of 1% wt, 5% wt, 10% wt, or between 5%-10% wt; phenol red with a wt % of one of 0.50% wt, 1% wt, 1.50% wt, 2.0% wt, between 0.50%-1.0% wt, between 1.0%-1.5% wt, or between 1.5%-2.0% wt; common yellow pigment with a wt % of one of 1% wt, 1.50% wt, 2.0% wt, 2.50% wt, between 1%-1.50% wt, between 1.5%-2.0% wt, between 2.0%-2.50% wt; common blue pigment with a wt % of one of 0.50% wt, 1% wt, 1.50% wt, 2.0% wt, between 0.50%-1.0% wt, between 1.0%-1.5% wt, between 1.5%-2.0% wt; and quaternary ammonium bromide with a wt % of one of 0.50% wt, 1% wt, 1.50% wt, 2.0% wt, between 0.50%-1.0% wt, between 1.0%-1.5% wt, between 1.5%-2.0% wt. In some embodiments, the IORC-01 ink includes styrene acrylic emulsion resin with a wt % of one of 50% wt, 60% wt, 70% wt, 80% wt, 90% wt, between 50%-60% wt, between 60%-70% wt, between 70%-80% wt, between 80%-90% wt; water with a wt % of one of 8% wt, 10% wt, 12% wt, 14% wt, 16% wt, 18% wt, 20% wt, between 5%- 10% wt, between 10%-15% wt, between 15%-20% wt; rosolin acid with a 1% wt, 2% wt, 3% wt, 4% wt, 5% wt, 6% wt, 7% wt, 8% wt, 9% wt, 10% wt, between 1%-3% wt, between 3%-5% wt, between 5%-7% wt, between 7%-10% wt, between 0%-5% wt, between 5%-10% wt; and petroleum defoamer with a wt % of one of 0.50% wt, 1% wt, 1.50% wt, 2.0% wt, between 0.50%-1.0% wt, between 1.0%-1.5% wt, between 1.5%-2.0% wt.

In some embodiments, the IORC-01 ink comprises a 88.5% wt of styrene acrylic emulsion resin, a 8.5% wt of water, a 1% wt of rosolin acid, and a 1% wt of petroleum defoamer.

### Method of Manufacture

To manufacture the CI strip, the IEVG-02 is first printed and then dried onto a film substrate. The IORC-01 ink is then used and printed on top of the IEVG-02 ink and allowed to dry before use.

### In Operation

As discussed above, the indicator of the CI strip is printed with a IEVG-02 ink and a IORC-01 ink. In some embodiments, as shown in FIGS. 1A-1C, when a threshold concentration of H₂O₂ and ozone (e.g., activated oxygen) is present, and the time limit of exposure is reached, the indicator will change from a red/orange color to a green color.

In some examples, the chemical indicator can change its color when all or most of the sensitive chemicals and inks on the indicator surface reacts with the active molecules (e.g., H₂O₂ and O₃) in the circulating effluent. In some embodiments, active molecules are delivered to the surface of the indicator through diffusion between the surface and the effluent in the gas form. In some examples, the total amount of delivered active molecules (R) to the surface is a product of the delivery rate (K) and exposure time (T). In some embodiments, K can be expressed as:

K=D*C;

D=diffusion coefficient, C=active molecule concentration

Total active molecule delivery=R=K*T=D*C*T

In some examples, the indicator color change occurs when the concentration of active molecules and exposure time is sufficient to cause the surface chemicals to react. This can indicate that the disinfection parameters were met. In some embodiments, the exposure time can be 5 minutes, 6 minutes, 7 minutes, 8 minutes, 9 minutes, 10 minutes, 11 minutes, 12 minutes, 13 minutes, 14 minutes, 15 minutes, between 5 minutes-6 minutes, between 6 minutes-7 minutes, between 7 minutes-8 minutes, between 8 minutes-9 minutes, between 10 minutes-11 minutes, between 11 minutes-12 minutes, between 12 minutes-13 minutes, between 13 minutes-14 minutes, between 14 minutes-15 minutes, and any value in between those ranges listed, including endpoints. In some embodiments, the chemistry of the CI strip can be customized to cycle time.

In some embodiments, this color change is brought about by a reaction of the H₂O₂ with the bromide compound in the IEVG-02 ink. In some examples, the color change is brought about by a reaction of the given concentration H₂O₂ for a minimum prescribed time with the bromide compound in the IEVG-02 ink. This can produce HBR followed by a redox reaction that impacts the phenol red in the IEVG-02 ink to produce a colorless end color. This then allows the two pigments of yellow and blue in the IEVG-02 ink to show a green color.

In some examples, a second reaction occurs wherein the presence of ozone oxidizes the rosolin acid in the IORC-01 ink. The initial color of the rosolin acid is a red/orange color and, once oxidized, it becomes colorless. The combination of the two reactions—the reaction of the H₂O₂ with the IEVG-02 ink and the reaction of the ozone (e.g., activated oxygen) with the IORC-01 ink—produces a green signal color when the two inks are printed on one another. In some embodiments, the reaction is accomplished by the minimum prescribed time.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention. The drawings are for the purpose of illustrating embodiments of the invention only, and not for the purpose of limiting it.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “deploying an instrument sterilized using the systems herein” include “instructing the deployment of an instrument sterilized using the systems herein.” In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 10 nanometers” includes “10 nanometers.”

Any titles or subheadings used herein are for organization purposes and should not be used to limit the scope of embodiments disclosed herein.

1. A chemical indicator strip for detecting hydrogen peroxide and/or ozone, wherein the chemical indicator strip comprises:
a film substrate;
an indicator portion comprising a first ink and a second ink; and
wherein the first ink and the second ink react in the presence of a threshold level of disinfectant for a threshold level of exposure time.

2. The chemical indicator strip of claim 1, wherein the first ink and the second ink are water based resin carriers.

3. The chemical indicator strip of claim 2, wherein the first ink and the second ink include an acrylic emulsion, water, reactive pigments, a non-reactive colorant, and a defoaming agent.

4. The chemical indicator strip of claim 1, wherein the first ink is IEVG-02 and wherein the second ink is IORC-01.

5. The chemical indicator strip of claim 4, wherein the IEVG-02 comprises a styrene resin, water, petroleum, ammonia, phenol red pigment, common yellow pigment, common blue pigment, and quaternary ammonium bromide.

6. The chemical indicator strip of claim 5, wherein the styrene resin comprises between a 60% wt-80% wt of the IEVG-02, wherein the water comprises between a 1% wt-15% wt of the IEVG-02, wherein the petroleum defoamer comprises between 0.5% wt-1.0% wt, between 1.0% wt-1.5% wt, or between 1.5% wt-2.0% wt of the IEVG-02, wherein the ammonia comprises between 0% wt-5% wt or between 5% wt-10% wt of the IEVG-02, wherein the phenol red pigment comprises between 0.5% wt-1.0% wt of the IEVG-02, wherein the common yellow pigment comprises between 1% wt-5% wt of the IEVG-02, wherein the common blue pigment comprises between 1.0% wt-1.5% wt of the IEVG-02, and wherein the quaternary ammonium bromide comprises between 6% wt-8% wt of the IEVG-0.

7. (canceled)

8. (canceled)

9. (canceled)

10. (canceled)

11. (canceled)

12. The chemical indicator strip of claim 5, wherein the IEVG-02 comprises styrene resin with a 70% wt, water with a 12% wt, petroleum with a 1.5% wt, ammonia with a 4% wt, phenol red pigment with a 1% wt, common yellow pigment with a 3% wt, common blue pigment with a 1% wt, and a quaternary ammonium bromide with a 7.5% wt.

13. (canceled)

14. The chemical indicator strip of claim 4, wherein the IORC-01 comprises styrene acrylic emulsion resin, water, rosolin acid, and petroleum defoamer.

15. The chemical indicator strip of claim 14, wherein the styrene acrylic emulsion resin comprises between 80% wt-90% wt of the IORC-01, wherein the water comprises between 5% wt-10% wt of the IORC-01, wherein the rosolin acid comprises between 0% wt-5% wt of the IORC-01, and wherein the petroleum defoamer comprises between 0% wt-2% wt of the IORC-01.

16. The chemical indicator strip of claim 14, wherein the IORC-01 comprises styrene acrylic emulsion resin with a 88.5% wt, water with a 8.5% wt, rosolin acid with a 1% wt, and petroleum defoamer with a 1% wt.

17. The chemical indicator strip of claim 1, wherein the film substrate comprises a single layer of biaxially oriented polypropylene.

18. The chemical indicator strip of claim 1, wherein the film substrate comprises an adhesive to allow the chemical indicator strip to attach to a surface.

19. The chemical indicator strip of claim 1, wherein the chemical indicator strip has a length between 2-4 inches and a width of between 0.75-1 inches.

20. The chemical indicator strip of claim 1, wherein the indicator portion is configured to change from a first color to a second color.

21. The chemical indicator strip of claim 20, wherein the first color is red or orange and wherein the second color is green.

22. (canceled)

23. The chemical indicator strip of claim 1, wherein the indicator portion is positioned at a first end of the chemical indicator strip.

24. The chemical indicator strip of claim 1, wherein the indicator portion is a square.

25. The chemical indicator strip of claim 20, wherein the indicator portion completely changes from the first color to the second color when the threshold level of disinfectant is detected.

26. The chemical indicator strip of claim 20, wherein the indicator portion does not change, or only partially changes from the first color to the second color when the threshold level of disinfectant is not detected.

27. (canceled)

28. The chemical indicator strip of claim 1, wherein the threshold level of exposure time is between 5 minutes and 10 minutes.

29-54. (Cancelled)
